(19) United States
(12) Patent Application Publication
(10) Pub. No.: US 2018/0140428 A1
(43) Pub. Date: May 24, 2018

(54) PATELLAR IMPLANTS AND TRIALS

(71) Applicant: Zimmer, Inc., Warsaw, IN (US)

(72) Inventor: David Brown, Warsaw, IN (US)

(21) Appl. No.: 15/815,373

(22) Filed: Nov. 16, 2017

Related U.S. Application Data
(60) Provisional application No. 62/424,013, filed on Nov. 18, 2016.

(51) Int. Cl. A61F 2/38; A61F 2/46; A61F 2/30

(52) U.S. Cl. CPC A61F 2/3877; A61F 2/4657; A61F 2/30771; A61B 17/14; A61F 2/4684; A61F 2002/4661; A61F 2002/30934

(57) ABSTRACT

Methods and apparatuses including a patellar implant are disclosed. The patellar implant can include a member, an articulation component and a connection mechanism. The member can be configured to engage a osteotimized surface of a patella. The articulation component can have an articulation surface that can be configured to articulate with a femoral prosthesis along a patellar groove. The connection mechanism can connect the articulation component with the member and can be configured to allow for at least one of removal and translational movement of the articulation component relative to the member and patella.

### CLAIM OF PRIORITY

This application claims the benefit of priority to U.S. Provisional Application Ser. No. 62/424,013, filed on Nov. 18, 2016, which is incorporated herein by reference in its entirety.

### FIELD

The present subject matter relates to orthopedic procedures and, more particularly, to patellar apparatuses for knee arthroplasties.

### BACKGROUND

The knee joint is generally formed by the pair of condyles located at the distal portion of a femur, the tibial plateau located at the proximal end of a tibia and a pair of menisci positioned between the tibial plateau and the femoral condyles. The knee further includes the patella which is secured by the patellar tendon to ride against an anterior portion of the femur during articulation of the knee.

Orthopedic procedures and prostheses are commonly utilized to repair and/or replace damaged bone and tissue in the human body. For example, a knee arthroplasty can be used to restore natural knee function by repairing damaged or diseased articular surfaces of the femur and/or tibia. In knee arthroplasty, portions of the natural knee joint are replaced with prosthetic components. These components include a tibial component, a femoral prosthesis, and a patellar component. The femoral prosthesis include prosthetic condyles that articulate with the tibial component and the femoral prosthesis form a patellar groove between the condyles, which is the articulating surface in which the patellar component moves,

Various types of patellar components having different sizes and geometry are known. For example, the Zimmer Natural Knee II System, includes a series of differently sized circular patellar prostheses. Patellar components with spherical and conically shaped articulation surface geometry can also be utilized depending upon the knee system selected.

### Overview

The present inventors recognize, among other things, an opportunity for improving patello-femoral joint balance and kinematics so as to improve patient satisfaction. More particularly, the present inventors have recognized that most current patello-femoral joint restoration techniques tend to focus on restoring a thickness of the patella. As such, these techniques do not match osteotomy of the patella to account for osteotomy of the femur as well.

Furthermore, the present inventors have recognized that patello-femoral joint balance and kinematics can be improved with various patellar implants disclosed herein. More particularly, the present inventors have developed patellar implants with various geometries and construction that allow the patellar implants to better articulate along the patellar groove. For example, the present inventors have disclosed herein patellar implants that compensate for side-to-side (medial-lateral) and/or rotational movement of the patellar implant along the patellar groove. The present inventors also disclose tilted osteotomy of the patella and tilted and/or offset patellar implants or modular inserts that can be used to produce shifted articulation of the patellar implant along the patellar groove. Constructs for trial patellar implants allowing for rapid and simplified swapping between various thicknesses of patellar implants is also disclosed.

As used herein the term “implant” or “prosthesis” includes both temporary trial implants/prostheses as well as implants/prostheses configured to be permanently implanted on the patella. Similarly the terms “osteotimize” “osteotimized” “osteotimizing” or similar are used synonymously with the terms “resect” “resected” “resecting” or the like.

To further illustrate the apparatuses and methods disclosed herein, the following non-limiting examples are provided:

Example 1 is a patellar implant that can comprise: a member configured to engage a osteotimized surface of a patella; an articulation component having an articulation surface configured to articulate with a femoral prosthesis along a patellar groove; and a connection mechanism connecting the articulation component with the member, the connection mechanism configured to allow for at least one of removal and translational movement of the articulation component relative to the member and patella.

In Example 2, the subject matter of Example 1 optionally includes the connection mechanism can be further configured to allow for rotational movement of the articulation component relative to at least one of the member and patella.

In Example 3, the subject matter of any one or more of Examples 1-2 optionally can include the member defines a slot configured to receive the connection mechanism therein, the slot configured to extend generally medial-lateral along the member from a first end to a second end when the member is connected to the patella.

In Example 4, the subject matter of any one or more of Examples 1-3 optionally can include the articulation surface is symmetrically shaped as at least one of a spherical dome, a contoured dome or a conical dome.

In Example 5, the subject matter of any one or more of Examples 1-4 optionally can include the connection mechanism comprises one or more finger projections extending from an edge of the articulation component, and wherein the member defines one or more grooves configured to receive the one or more finger projections therein.

In Example 6, the subject matter of Example 5 optionally can include the articulation component comprises a trial prosthetic and is one of a plurality of articulation components, each of the plurality of articulation components configured to attachable and detachable in a substitutable manner, each of the plurality of articulation components configured to create a different thickness for the patellar implant when attached.

In Example 7, the subject matter of any one or more of Examples 1-6 optionally can include the connection mechanism comprises a post extending from an opposing side of the articulation component from the articulation surface, and wherein the member defines a slot having a plurality of ridges configured to receive the post therein.

Example 8 is a patellar implant that can comprise: an articulation component having an articulation surface configured to articulate with a femoral prosthesis at a patello-femoral joint; a member configured to engage a osteotimized surface of a patella, the member configured to connect to the articulation component; wherein the articulation component is non-symmetrically shaped about a geometric center of the articulation component and is configured for rotational movement about the member and relative to the patella and the femoral prosthesis to create a variable angle relative to a sulcus of the femoral prosthesis.

In Example 9, the subject matter of Example 8 optionally can include wherein at least one of the member, a modular component and the articulation component are configured to allow for translational movement of the articulation component relative to the member, the femoral prosthesis and the patella.

In Example 10, the subject matter of any one or more of Examples 8-9 optionally can include a second articulation component configured to connect to and cover the articulation component and interface with the femoral component.

Example 11 is a method of preparing a knee joint for a patellar implant, the method can comprise: measuring a position of a patella within the knee joint including measuring a thickness of the patella; resecting a femur; measuring an amount of femur removed by resecting the femur from an anterior chamfer and further adding a thickness of the saw blade to the amount to obtain a first patellar groove thickness; implanting a desired size of femoral prosthesis on the femur after resecting the femur; measuring a thickness of the femoral prosthesis in a same location of the anterior chamfer to obtain a second patellar groove thickness; determining an amount of bone to be removed from the patella by compensating for the difference between the first patellar groove thickness and the second patellar groove thickness; and after determining the amount of bone to be removed from the patella, resecting the patella to an appropriate thickness to receive the patella implant.

In Example 12, the subject matter of Example 11 optionally can include determining an desired thickness for the patellar implant based upon kinematic tests including a range of motion of a trial implant with along a patello-femoral joint between the trail implant and the femoral prosthesis, wherein the trial implant includes an articulation component that is configured to be attachable to and detachable from a member thereof.

In Example 13, the subject matter of any one or more of Examples 11-12 optionally can include wherein resecting the patella includes performing an osteotomy of the patella to form a substantially planar osteotimized surface that is angled medial-to-lateral such that at least one of a thickness of the patella at a medial edge is greater than a thickness of the patella at a lateral edge or the thickness of the patella at the medial edge is greater than the thickness of the patella at the lateral edge.

In Example 14, the subject matter of any one or more of Examples 11-13 optionally can further comprise: coupling the patellar implant to the patella, the patellar implant comprising: a member configured to engage a osteotimized surface of a patella; an articulation component having an articulation surface configured to articulate with a femoral prosthesis along a patellar groove; and a connection mechanism connecting the articulation component with the member, the connection mechanism configured to allow for at least one of removal and translational movement of the articulation component relative to the member and patella.

In Example 15, the subject matter of Example 14 optionally can include the patellar implant is configured to allow for rotational movement of the articulation component relative to at least one of the member and patella.

In Example 16, the subject matter of any one or more of Examples 11-15 optionally further comprises: coupling the patellar implant to the patella, the patellar implant comprising: an articulation component having an articulation surface configured to articulate with a femoral prosthesis at a patello-femoral joint; a member configured to engage a osteotimized surface of a patella, the member configured to connect to the articulation component; wherein the articulation component is non-symmetrically shaped about a geometric center of the articulation component and is configured for rotational movement about the member and relative to the patella and the femoral prosthesis to create a variable angle relative to a sulcus of the femoral prosthesis.

In Example 17, the subject matter of Example 16 optionally can include at least one of the member, a modular component and the articulation component are configured to allow for translational movement of the articulation component relative to the member, the femoral prosthesis and the patella.

In Example 18, the subject matter of any one or more of Examples 16-17 optionally can include a second articulation component configured to connect to and cover the articulation component and interface with the femoral component.

Example 19 is a method of preparing a knee joint for a patellar implant, the method comprising: measuring a position of a patella within the knee joint including measuring a thickness of the patella; resecting a femur; resecting the patella; after resecting the patella, selecting the femoral prosthesis and the patellar implant that provide a desired total thickness for the combination of the femoral prosthesis and the patellar implant; wherein the desired total thickness is determined by: measuring an amount of femur removed by resecting the femur from an anterior chamfer and further adding a thickness of the saw blade to the amount to obtain a first patellar groove thickness; measuring a thickness of the femoral prosthesis in a same location of the anterior chamfer to obtain a second patellar groove thickness; selecting a thickness of the patellar implant to an amount of bone resected from the patella as well as any difference between the first patellar groove thickness and the second patellar groove thickness.

In Example 20, the apparatuses or method of any one or any combination of Examples 1-19 can optionally be configured such that all elements or options recited are available to use or select from.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatuses and methods.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various examples discussed in the present document.

FIG. 1 is an anterior view of a portion of a knee joint illustrating the patella in an everted position to allow for preparation of a posterior surface thereof along with a system of preparation tools including a cutting instrument and patella sizer according to an example of the present application.

FIG. 1A is an enlarged view of the patella sizer of FIG. 1 according to an example of the present application.

FIG. 2 is an anterior view of the patella after an initial preparation with a patellar implant mounted thereto according to an example of the present application.

FIG. 3 is a perspective view of the patellar implant of FIG. 2 with the anatomical features shown in FIG. 2 removed to show a member and an articulation component according to an example of the present application.

FIGS. 4A-4D are views of the member of FIG. 3 from various perspectives according to an example of the present application.

FIGS. 5A-5C are views of the articulation component and a connection mechanism from various perspectives according to an example of the present application.

FIG. 6 is a perspective view of a system that includes the patellar implant of prior FIGURES and a second articulation component according to an example of the present application.

FIG. 7 is a perspective view of the components of the system of FIG. 6 assembled together according to an example of the present application.

FIGS. 8A-8C are views of the second articulation component of FIGS. 6 and 7 from various perspectives according to an example of the present application.

FIG. 9 is an elevated perspective view of a femoral prosthesis interfacing with the patellar implant of prior FIGURES, the articulation component of the patellar implant positioned for medial-lateral movement along a sulcus of the femoral prosthesis according to examples of the present application.

FIG. 10 is an anterior view of the portion of the knee joint illustrating the patella in an everted position to allow for further preparation of a posterior surface thereof along with a system of preparation tools including a drill according to an example of the present application.

FIGS. 11 and 12 are perspective views of a second patellar implant including a second articulation component and a second member according to an example of the present application.

FIG. 13 is a plan view of a side of a third patellar implant including a third articulation component that can be used with the second member according to an example of the present application.

FIGS. 14A and 14B are views of the third articulation component of the third patellar implant of FIG. 13 according to an example of the present application.

FIG. 15 is an elevated perspective view of the femoral prosthesis interfacing with the third patellar implant, the articulation component of the patellar implant positioned for rotational movement along a sulcus of the femoral prosthesis according to examples of the present application.

FIG. 16 is a plan view a fourth patellar implant including a fourth articulation component and a third member according to an example of the present application.

FIG. 17 is a cross-sectional view of the fourth articulation component according to an example of the present application.

FIG. 18 is a plan view of the third member according to an example of the present application.

FIG. 19 is an elevated perspective view of the femoral prosthesis interfacing with the fourth patellar implant including the first member, the articulation component of the patellar implant positioned for rotational and medial-lateral movement along a sulcus of the femoral prosthesis according to examples of the present application.

FIGS. 20 and 20A show views including a cross-section of the femoral prosthesis according to an example of the present application.

FIG. 21 shows a side view of a patella and one of the aforementioned patellar implants interfacing with the femoral prosthesis of FIGS. 20 and 20A according to an example of the present application.

FIG. 22A is a schematic elevated view of the patella relative to an anterior portion of the natural femur showing various thicknesses used in a osteotomy technique according to an example of the present application.

FIG. 22B is a schematic elevated view of the osteotimized patella and patellar implant relative to an anterior portion of the femoral prosthesis showing how the various thicknesses of FIG. 22A are translated after the osteotomy technique according to an example of the present application.

### DETAILED DESCRIPTION

The present application relates to devices and methods that can be used in a total knee replacement procedure (TKA) where the patello-femoral joint is replaced by a femoral prosthesis as well as a patellar implant connected to a osteotimized portion of a patient's patella. The disclosed patellar implants can be trial implants/prosthetics configured for temporary use to judge patello-femoral joint kinematics or permanent implants/prosthetics. Thus, as used herein the term “patellar implant”, “patellar component” or “patellar prosthesis” can mean both a trial or a permanent implant.

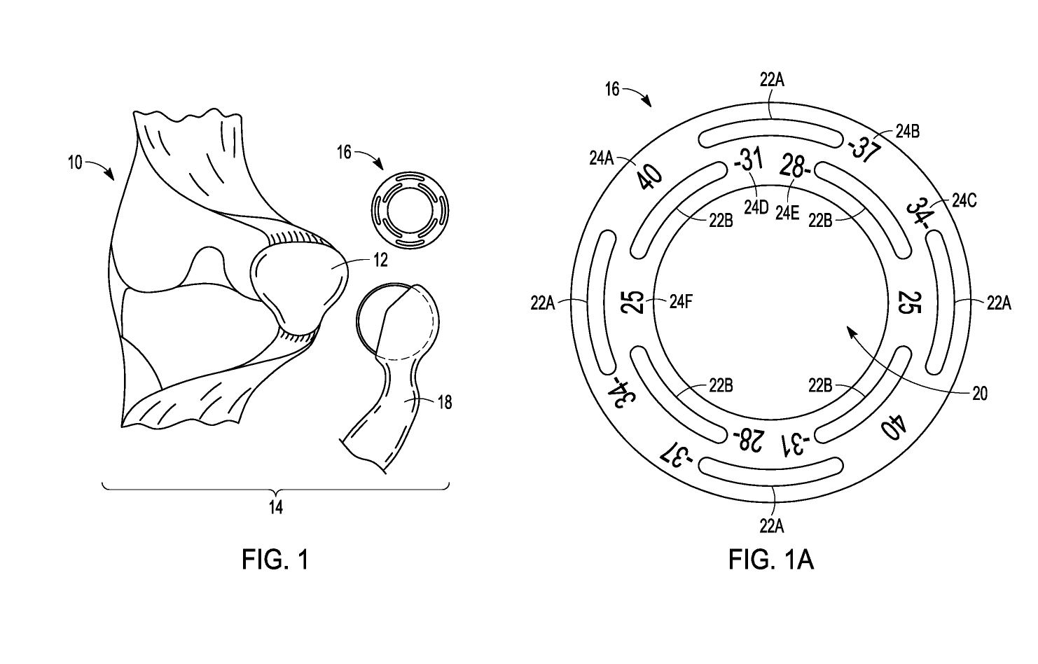

FIG. 1 shows a portion of a knee joint 10 of a patient. The knee joint 10 can include a patella 12. FIG. 1 also illustrates a system 14 including a patellar implant sizer 16 and a cutting instrument 18.

To prepare the patella 12 to receive a patellar implant, an incision is made to expose the knee joint 10 and the patella 12 can be everted as shown in FIG. 1. After everting patella 12, the cutting instrument 18 may be utilized to remove a posterior articular surface of patella 12 to form an osteotimized surface of patella 12. This osteotimized surface can be substantially planar according to some examples. In other examples, once the patella 12 is osteotimized the patella 12 can have a varying thickness medial-lateral for example. In other examples, the patella can have a substantially uniform thickness in the medial-lateral direction.

After osteotimizing the patella 12, the patellar implant sizer 16 can be placed at the osteotimized surface. In some examples, the patellar implant sizer 16 can be used subsequent to the use of a trial such as various of the patellar implants disclosed herein. In such cases, the patellar implant sizer can be used to indicate possible overhang of the patellar implant on the patella 12. Remedial steps can then be implemented such as the femur can be moved a small amount to adjust the patellar groove and thereby allow the patella 12 to be moved accordingly. According to further examples, the inner diameter of the central opening 20 can be configured to fit over an outer diameter of the patellar implants disclosed herein. This can allow the sizer to determine an optimal size of a permanent patellar implant to fit the patella 12 at a location determined by the trial. As shown in the enlargement of FIG. 1A, the patellar implant sizer 16 can comprise a thin ring shaped component comprised of a biocompatible material such as e.g., ultra high molecular weight polyethylene. The patellar implant sizer 16 can have a central opening 20 and a plurality of visualization slots 22A and 22B for visualization of the patella 12 when positioned on the patella 12 (FIG. 1). The patellar implant sizer 16 can include various reference markings 24A, 24B, 24C, 24D, 24E and 24F indicating specific stock sizes of patellar implant. In some examples, the central opening 20 can correspond to a specific smallest size of patellar implant and/or the outer rim of the patellar implant sizer 16 can correspond to a largest size of patellar implant.

As used herein, “proximal” refers to a direction generally toward the torso of a patient, and “distal” refers to the opposite direction of proximal, i.e., away from the torso of a patient. As used herein, the terms “anterior” and “posterior” should be given their generally understood anatomical interpretation. Thus, “posterior” refers to a rear of the patient, e.g., a back of the knee. Similarly, “anterior” refers to a front of the patient, e.g., a front of the knee. Thus, “posterior” refers to the opposite direction of “anterior”. Similarly, the terms “medial” and “lateral” should be given their generally understood anatomical interpretation. “Medial” refers to the opposite direction of “lateral”.

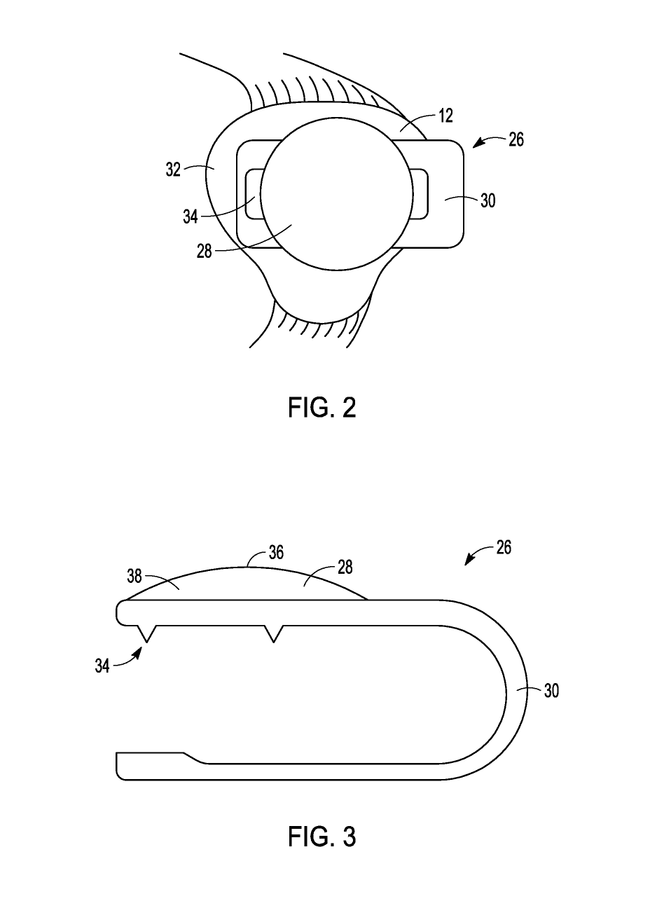

FIG. 2 shows the patella 12 after being osteotimized with a first patellar implant 26 coupled thereto. The construct of the first patellar implant is further illustrated in FIG. 3 with the patella 12 removed. As shown in FIGS. 2 and 3, the first patellar implant 26 can include a first articulation component 28 and a member 30.

In the example of FIG. 2, the first patellar implant 26 can be arranged such that the member 30 extends generally medial-lateral and can wrap around at least a portion of the patella 12. Indeed, the member 30 can be configured to engage the osteotimized surface of the patella 12. According to the example of FIG. 2, the portion of the patella 12 that can be wrapped by the member 30 can comprise three sides of the patella 12 with a fourth side 32 of the patella 12 un-covered. According to some examples, the fourth side 32 can comprise one of a medial or lateral side of the patella 12. However, the member 30 can be oriented to extend generally in any of a plurality of directions not just generally medial-lateral shown in FIG. 2. Thus, in some cases the fourth side 32 of the patella 12 can include medial and proximal, medial and distal, lateral and proximal and/or lateral and distal portions of the patella 12.

As shown in FIG. 2, the member 30 can be configured to form a slot 34 therein. The slot 34 can extend medial-lateral and can facilitate generally medial-lateral movement to reposition the articulation component 28 relative to the patella 12, the member 30 and a femoral prosthesis (now shown).

The construct of the articulation component 28 can be of any shape known in the art and further shapes disclosed herein. Thus, the articulation component 28 can be a patellar button having a domed shape (e.g., can be any one of spherically domed, conically domed, or contoured domed) according to some examples. The articulation component can have an articulation surface 38 configured to articulate with a femoral prosthesis along a patellar groove (i.e. along the sulcus which comprises an imaginary line connecting distal-most points of the patellar groove). As shown in FIG. 3, the articulation component 28 can have an apex 36 along the topmost point of articulation surface 38 thereof. The articulation component 28 and the member 30 can be formed of any biocompatible material including for example various polymers including ultra high molecular weight polyethylene, ceramic materials and metals such as stainless steel, titanium and cobalt chrome alloys.

As is shown in FIGS. 2 and 3, the articulation component 28 can be coupled to the member 30 via the slot 34. This can allow for translational constrained movement of the articulation component 28 along the slot 34 relative to the member 30. This constrained movement can be generally medial-lateral movement or movement in another direction as discussed previously.

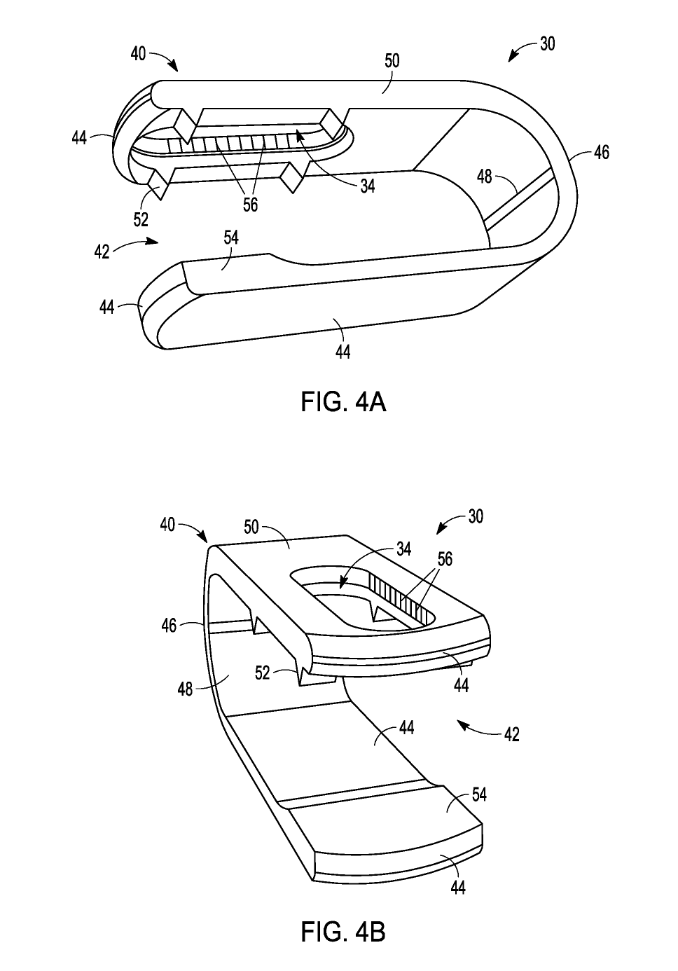

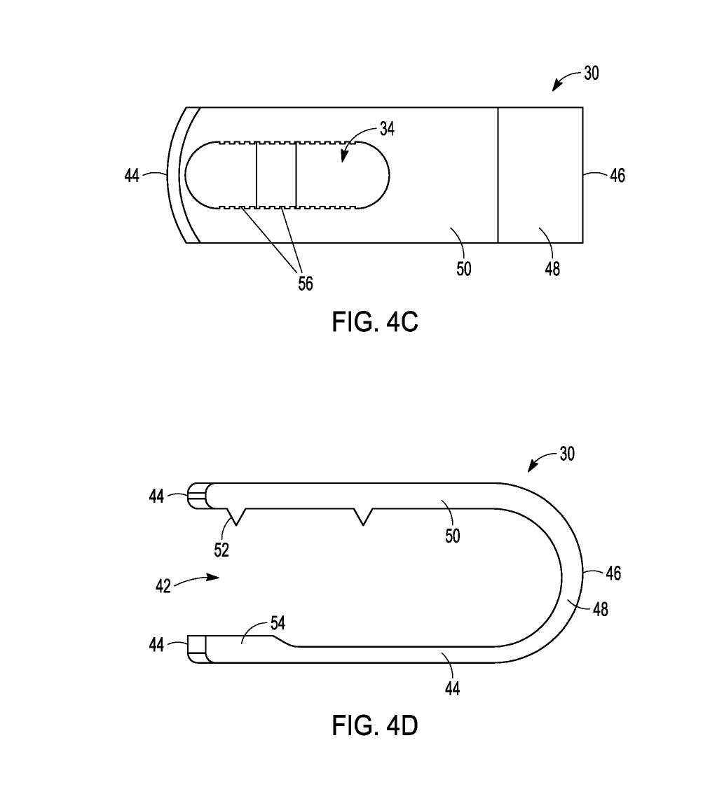

As shown in FIGS. 4A-4D, the member 30 can be configured as a clip 40 with an opening 42 at a first end 44. The clip 40 can have a base 44 extending from the first end to a second end 46. The second end 46 can have a U-shaped transition portion 48 that can extend between the base 44 and a top 50. The top 50 can include features such as the aforementioned slot 34, as well as retention features 52 (e.g., pegs) that extend from the top 50 toward the base 44. The retention features 52 can be configured to engage the patella 12 (FIG. 1) along a posterior thereof.

The base 44 can be configured to engage with the patella 12 (FIG. 1) along an anterior portion thereof. The clip 40 can be inserted in a first direction (e.g., medial-lateral) about the patella 12 so as to engage the patella 12 in the manner illustrated in FIG. 2. As such, the clip 40 can be somewhat flexible (e.g., the transition portion 48 can allow for movement of the top 50 and/or bottom 44 relative to one another. The clip 40 can be biased by the shape of the transition portion 48 and the materials selected to urge the top 50 and the base 44 to engage with the patella 12 (FIG. 2). Additionally, fixation features such as the retention features 52 and a lower lip 54 on the base 44 can be utilized to facilitate coupling of the clip 40 to the patella.

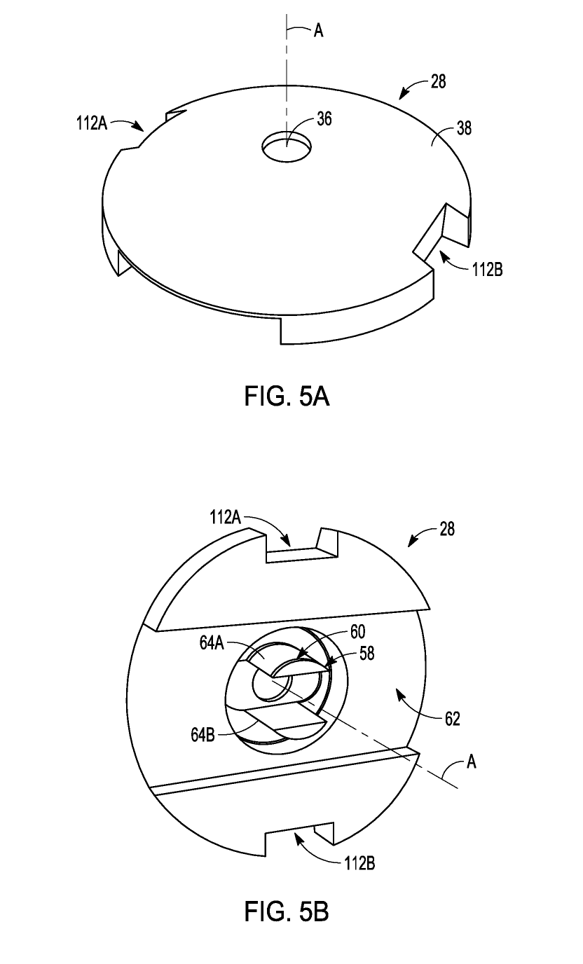

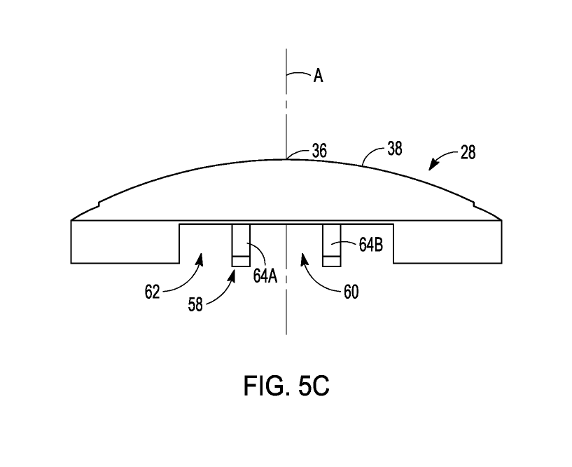

As shown in FIGS. 4A and 4B, the top 50 along the slot 34 can include a plurality of ridges 56. The plurality of ridges 56 can be configured to provide discrete locations to position the articulation component 28 (FIGS. 2 and 3) relative to the clip 40 and other anatomy of the patient. More particularly, the plurality of ridges 56 can be configured to engage with a connection mechanism (shown in FIGS. 5B and 5C) to couple the articulation component 28 (FIGS. 2 and 3) relative to the member 30 (e.g., the clip 40).

FIGS. 5A-5C further illustrate an example of the articulation component 28 as well as the connection mechanism 58. As discussed previously, the articulation component 28 can be a patellar button having a domed shape (e.g., can be any one of spherically domed, conically domed, or contoured domed) according to some examples. In the example of FIGS. 5A-5C, the articulation component 28 has a spherical dome shape. The articulation component 28 can be symmetrically shaped about axis A and can have the articulation surface 38 configured to articulate with a femoral prosthesis along a patellar groove(i.e. along the sulcus thereof). As shown in FIG. 3, the articulation component 28 can have an apex 36 along the topmost point of articulation surface 38 that is aligned with the axis A of component symmetry.

The small diameter hole such as the one show at the center of the articulation surface 38 (at least a relative location in reference to FIGS. 5A-5C) can be used for locating a peg(s) of a permanent implant on the bone. The hole can move with the articulation as the trial articular component (for example can move medial-lateral side-to-side as it interacts with the femoral prosthesis (trial or implant). Once a desired position is confirmed to give good joint kinematics, the position for the peg(s) can be marked thru the hole (either by drill or marker). That location can be used to complete the peg preparation illustrated in subsequent FIGURES.

In FIGS. 5B and 5C, the connection mechanism 58 can be integrally formed with the articulation component 28. However, in other examples the connection mechanism 58 can be a separate component from the articulation component 28 as will be discussed and illustrated subsequently in further examples. As shown in FIGS. 5B-5C, the connection mechanism 58 can comprise a post 60 and groove 62. The post 60 can comprise two prongs 64A and 64B that can be configured to be somewhat flexible to snap engage with the plurality of ridges 56 shown in FIGS. 4A and 4B. The groove 62 can comprise a recessed portion of a bottom surface 66 of the articulation component 28 and can extend along an entire portion of the bottom surface 66. The groove 62 can be sized and shaped to receive the top 50 (FIGS. 4A-4D) of the clip 40 (FIGS. 4A-4D) therein. The groove 62 can be provided with some tolerance to allow for sliding movement of the articulation component 28 as previously discussed to reposition the articulation component 28 relative to the member 30 (e.g., the clip 40 of FIGS. 4A-4D) and other anatomy of the patient.

According to one example, the connection mechanism 58 can connect the articulation component 28 with the member 30 as previously described and illustrated. The connection mechanism 58 can be configured to allow for at least one of removal (described in subsequent examples) and translational medial-lateral movement of the articulation component 28 relative to the member 30 and patella as previously described.

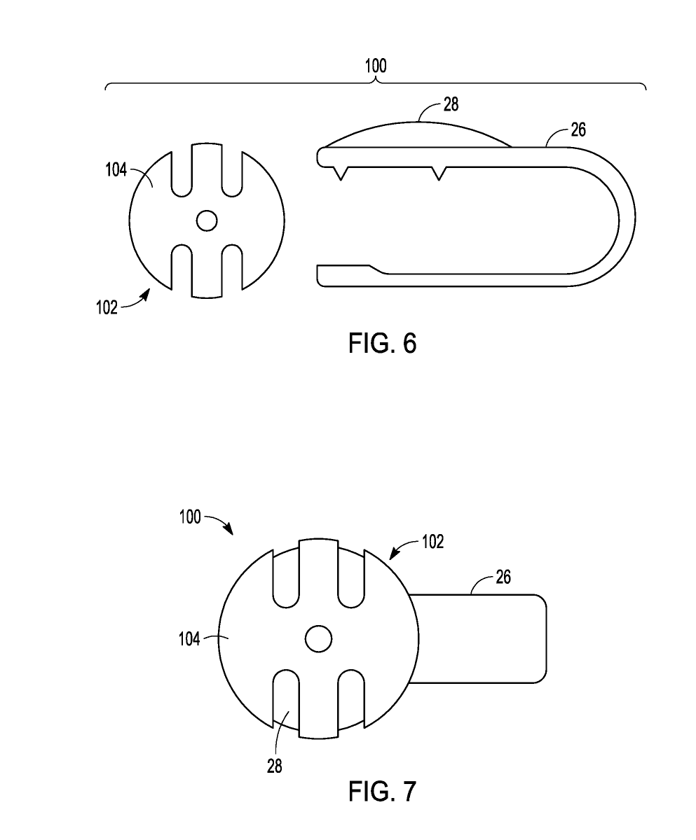

FIGS. 6 and 7 illustrate that in another example the first patellar implant 26 can be part of a system 100 that can further include a second articulation component 102. FIG. 6 shows the system 100 with the second articulation component 102 disassembled from the first patellar implant 26. FIG. 7 shows the system 100 with the second articulation component 102 assembled with the first patellar implant 26 and first articulation component 28. When assembled, the second articulation component 102 covers the first articulation component 28, and is configured with a similarly shaped articular surface 104 thereto. The articulation surface 104 is configured to interface with a femoral prosthesis.

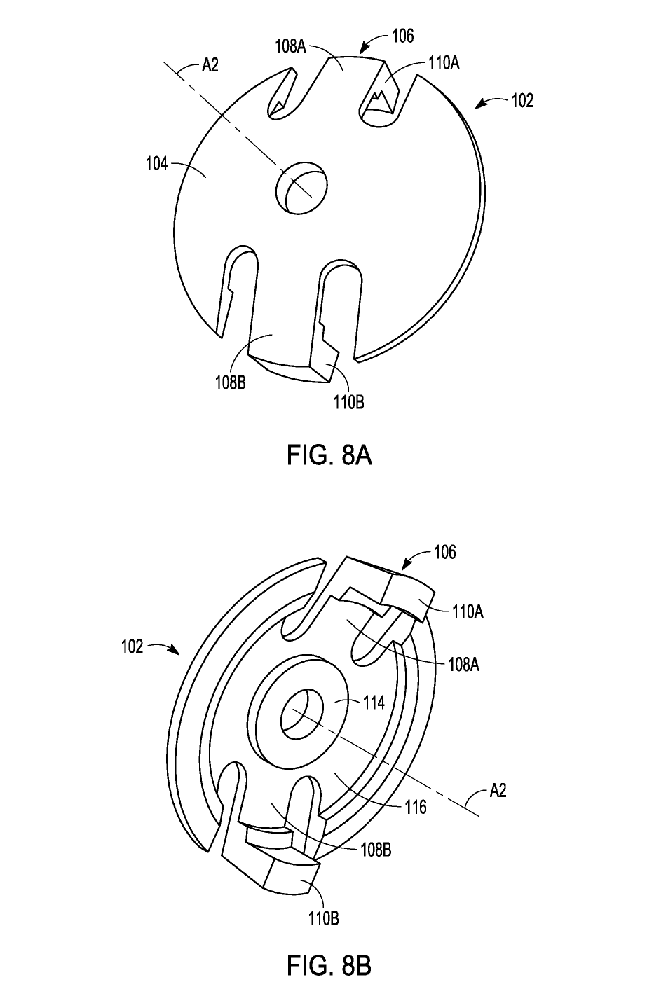

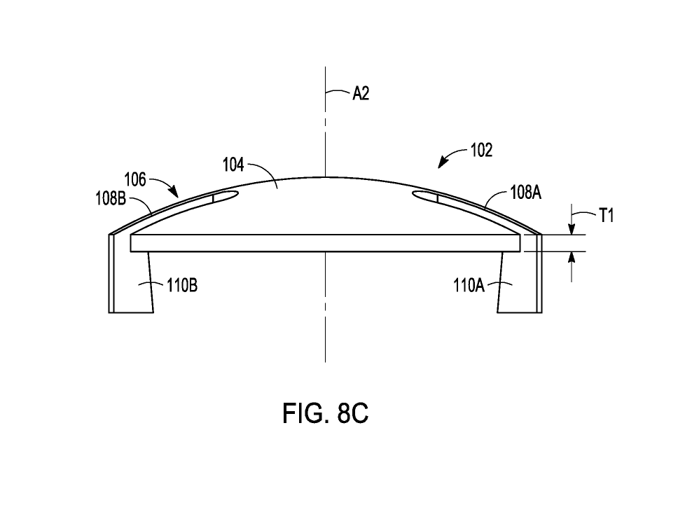

The second articulation component 102 can comprise a trial component configured to add an additional thickness to the first articulation component 28. Thus, the second articulation component 102 can be provided in a variety of stock thicknesses (e.g., 2 mm, 4 mm, 6 mm, etc.) as part of the system 100. FIG. 8C shows the second articulation component 102 with a first thickness T1. In use, a physician can perform kinematic tests on the knee joint of the patient with and/or without the second articulation component 102. If tests indicate that the thickness of the first patellar implant 26 needs to be increased as measured anterior-posterior than one of the second articulation components can be coupled to the first patellar implant 26 until a desired thickness can be achieved. Thus, according to one example, the second articulation component 102 can comprise a trial prosthetic that can be one of a plurality of articulation components. Each of the plurality of articulation components can be configured to attachable and detachable in a substitutable manner to the first articulation component 28 (FIG. 6) or another component such as the member, baseplate or the like. Each of the plurality of articulation components can be configured to create a different thickness for the patellar implant 26 when attached.

FIGS. 8A-8C show an example of the second articulation component 102 in further detail. The second articulation component 102 can be symmetrically shaped about axis A2 as shown in FIGS. 8A-8C. In the example of FIGS. 8A-8C, the articulation surface 104 can have a substantially similar shape to the articulation surface 38 of the first articulation component 28 (e.g., a spherical dome shape). However, in other examples, the shape of the articulation surface 104 can differ from that of the articulation surface 38 (e.g., articulation surface 104 can be conically domed, contoured dome or another shape while the articulation surface 38 can be spherically domed). Thus, in some cases the second articulation component 102 can be configured such that the articulation surface 104 can differ from that of the first articulation component 28.

As shown in the example of FIGS. 8A-8C, the second articulation component 102 has a connection mechanism 106 comprising two tabs 108A and 108B with fingers 110A and 110B extending therefrom. Each tab 108A and 108B extends radially outward from adjacent the axis A2 and is separated from the remainder of the second articulation component 102 by recesses/space as shown in FIG. 8B. The tabs 108A and 108B can be configured (shaped and constructed of particular materials) to be flexible proximal-distal. The finger 110A extends generally proximal-distal as well as radially inward toward axis A2 from outer radial end of tab 108A. Similarly, the finger 110B extends generally proximal-distal as well as radially inward toward axis A2 from outer radial end of tab 108B. The fingers 110A and 110B can be configured to engage with external grooves 112A and 112B, respectively in the first articulation component 28 as shown in FIGS. 5A and 5B.

FIG. 8B also illustrates the second articulation component 102 can be provided with a standoff feature 114 comprising a projection configured to extend from a backside surface 116 of the second articulation component 102. The standoff feature 114 can be configured to interface with and abut the articulation surface 38 of the first articulation component 28 when the second articulation component 102 is mounted thereto.

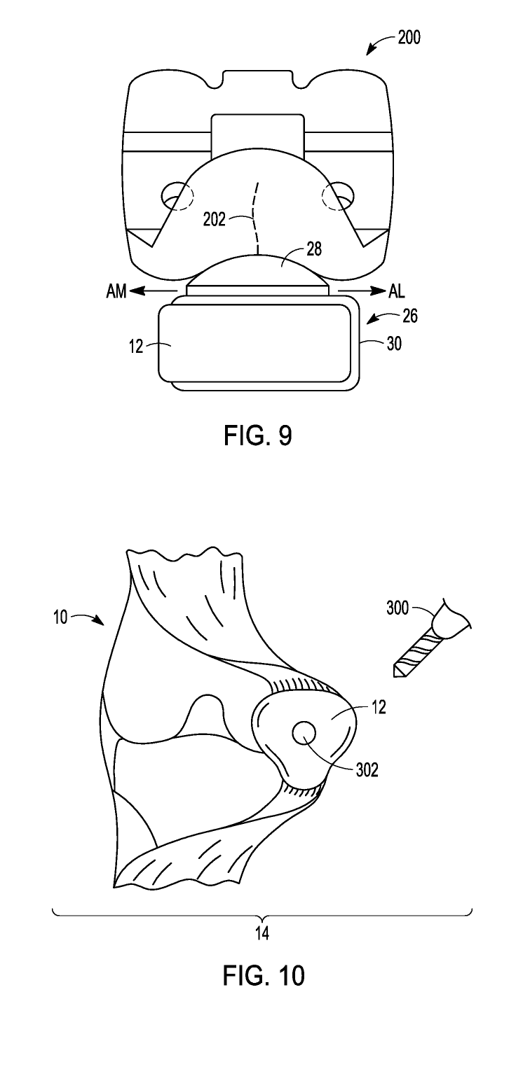

FIG. 9 shows a femoral prosthesis 200 interfacing with the patellar implant 26. The patellar implant 26 is mounted to a osteotimized patella 12. The first articulation component 28 of the patellar implant 26 can be positioned for medial-lateral movement (indicated by arrows AM and AL) along a sulcus 202 (indicated as a dashed line) of the femoral prosthesis 200 utilizing the member 30 and slot/connection mechanism arrangement previously described herein. Such arrangement can allow for better knee joint kinematics and increased patient satisfaction as the patellar implant 26 in particular the articulation surface 38 is better able to track along the sulcus 202 (an imaginary line connecting distal-most points of the femoral prosthesis within the patellar groove). Additionally, the disclosed arrangement with the first articulation component 28 configured for medial-lateral movement along the sulcus 202 can reduced or eliminate the development of a quadriceps angle relative to bone, which can reduce stress on the patella 12.

FIG. 10 shows a portion of the knee joint 10 of the patient as previously described in reference to FIG. 1. Thus, the knee joint 10 can include a patella 12 and the system 14 of components previously described. FIG. 10 further illustrates that the system 14 in some examples can include a drill 300 or similar tool for forming one or more holes 302 in the patella 12. The system 14 can be used as previously describe to osteotimize the patella 12.

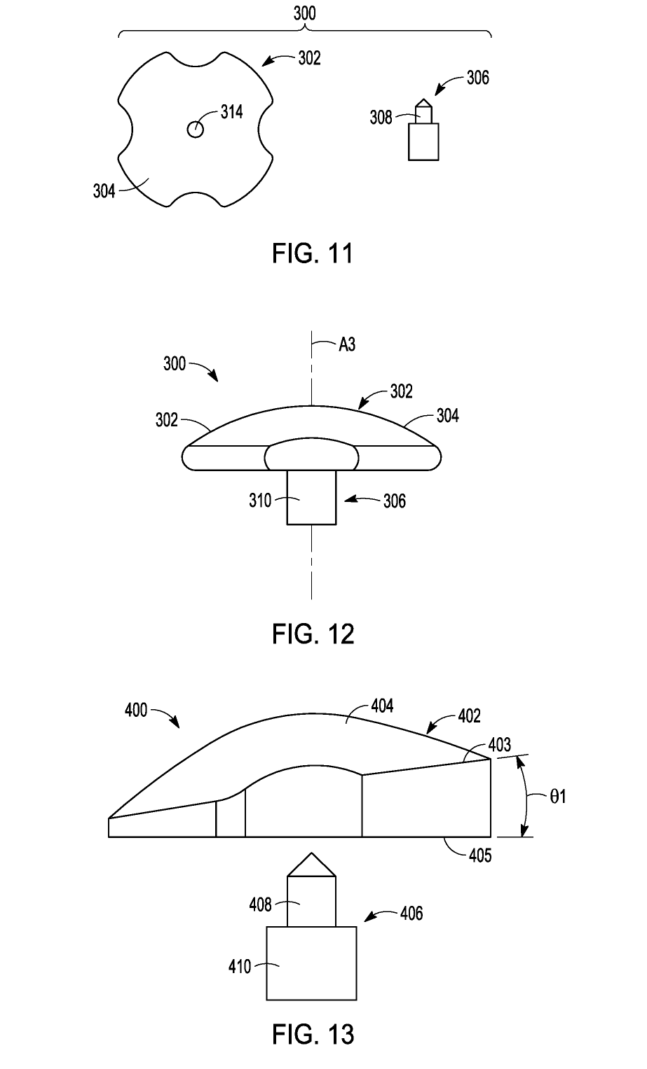

FIGS. 11 and 12 show a third patellar implant 300. FIG. 11 shows components of the third patellar implant 300 disassembled while FIG. 12 show the components assembled together. The third patellar implant 300 can include an articulation component 302 having a symmetrical construction of similar or identical articular surface 304 shape to those previously described. The third patellar implant 300 can also include a member 306 and a connection mechanism 308 (shown in FIG. 11 only). In the example of FIGS. 11 and 12, the member 306 can comprise a single fixation peg 310. The connection mechanism 308 can comprise a necked down portion of the peg 310 that is configured to be received in a passage 314 of the articulation component 302. The necked down portion can connect via interference fit. In some examples, adhesives or other connection forming materials can also be utilized to couple the member 306 to the articulation component 302.

As will be further illustrated and described in reference to FIGS. 13-15, the connection mechanism 308 and peg 310 can be configured to allow for rotational movement of the articulation component 302 about axis A3 (i.e. about the peg 310) of FIG. 12 relative to the femoral prosthesis and patella. Thus, the third patellar implant 300 can rotate relative to the patella about the peg 310 during kinematic range of motion testing once the peg 310 is implanted in the patella 12. This can allow the third patellar implant 300 to orient itself in or be turned to a kinematically advantageous position relative to the femoral implant and the patella.

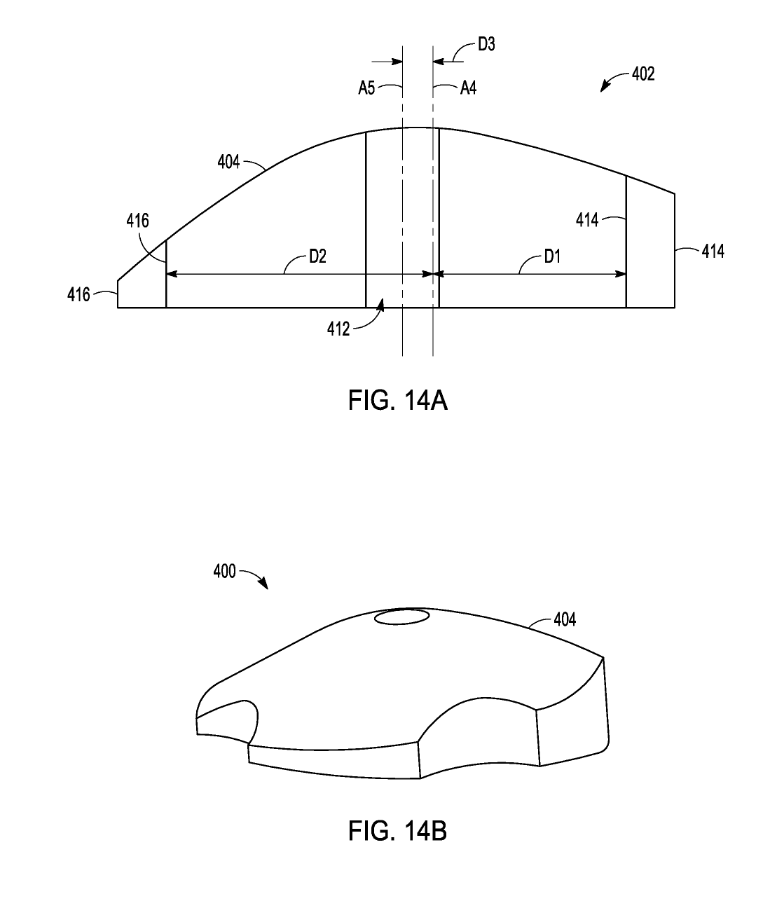

FIG. 13 shows a fourth patellar implant 400 that can include an articulation component 402 having an articulation surface 404. The fourth patellar implant 400 can also include a member 406 and a connection mechanism 408. As with the previously described example, the member 406 can comprise a peg 410 and the connection mechanism can be a necked down portion of the member 406. The connection mechanism 408 can be received in the passage 412 shown in FIG. 14A.

As shown in FIG. 13 the articulation component 402 is non-symmetrical in shape and can be configured to provide a tilt or angle θ1 to the articulation surface 404 as measured between an edge 403 of the articulation component and a base 405 in some examples. The articulation component 402 has a face center indicated by axis A4 as shown in FIG. 14A. The face center can comprise a plurality of points where of the articulation component 402 has substantially the same medial-lateral distances D1 and D2 as measured from the medial edge surface 414 and lateral edge 416, respectively. In some cases, the face center can align with the apex of the articulation surface 404. The face center as indicated by axis A4 can be offset a distance D3 from an articulation axis A5 of the articulation component 402. The articulation axis A5 can be defined as an axis passing along the passage 412 and through an axis of symmetry of the peg 410 about which the articulation component 402 can revolve when implanted on the patella.

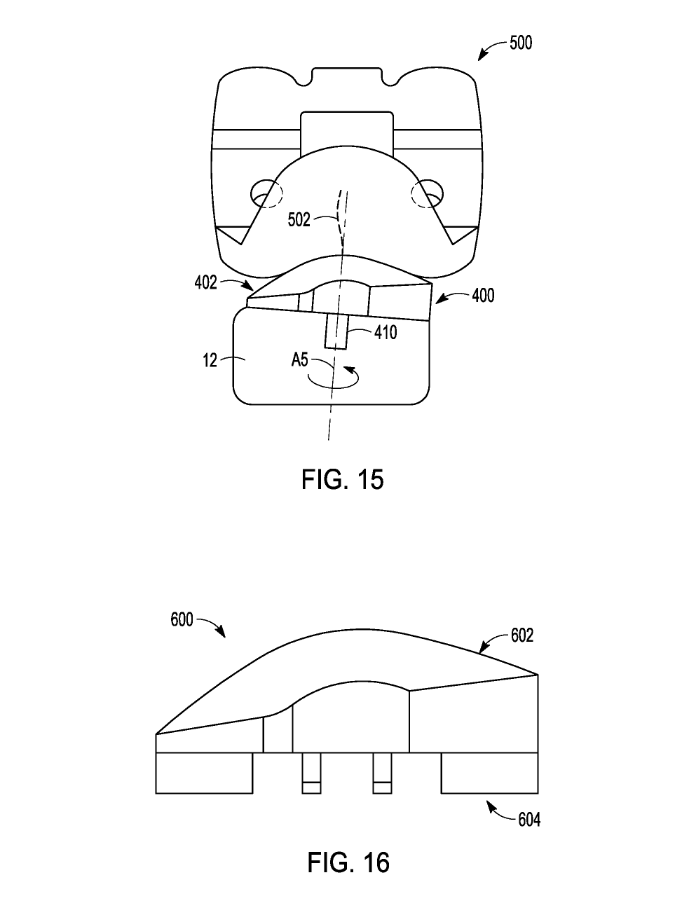

Similar to the example of FIGS. 11-12, the fourth patellar implant 400 can be configured for rotational movement relative to the femoral prosthesis and patella when implanted. The second articulation component of FIG. 8 (with modification to match the articular surface shape) can be used to add additional thickness to the fourth patellar implant 400. Furthermore, the patellar sizer discussed in reference to FIGS. 1 and 1A can also be used with the fourth patellar implant 400 as previously discussed. Rotational movement of the fourth patellar implant 400 is illustrated in FIG. 15 relative to a femoral prosthesis 500 and patella 12. Thus, the fourth patellar implant 400 can rotate relative to the patella 12 about the peg 410 during kinematic range of motion testing once the peg 410 is implanted in the patella 12. Similar to the third patella implant 300, the fourth patella implant 400 can ride in sulcus 502 (indicated as a dashed line) of femoral prosthesis 500 via rotation about the peg 410 during articulation of the knee. More particularly, the nonsymmetrical shape of the articular component 402 can allow for various angles relative to the femoral prosthesis 500 and sulcus 502 when rotated as the articular component 402 is capable of 360 degrees of rotation on the peg 410. The tilt or angle created by non-symmetric shape of the articular component 402 can result in various angles being created between the resected distal femur and the patella 12 while maintaining the articular surface orientation of the articular component 402 against the sulcus 502 as the articular component 402 is rotated about axis A5 (e.g., from 0 degrees anywhere to 360 degrees). This configuration can allow for desirable patellar tracking. The various angles can allow for proper articulation of the patellar implant even if the patellar bone is resected at a medial-lateral and an anterior-posterior angle. In such instances the patellar implant can be rotated so that the back face of the patellar implant allows the patella to orient as it was cut and still have the articulation surface aligned in a kinematically desirable manner.

According to one example, during kinematic range of motion testing of the knee joint, the fourth patellar implant 400 can rotate about the axis A5 defined by peg 410 as schematically illustrated in FIG. 15. Such rotation may occur until the fourth patellar implant 400 has aligned itself in a kinematically favored position. If the fourth patellar implant 400 is a trial, once the kinematically favored position is achieved, the patella 12 can be marked to reflect the rotational orientation of the so that a permanent prosthesis may be similarly oriented and affixed to the patella 12.

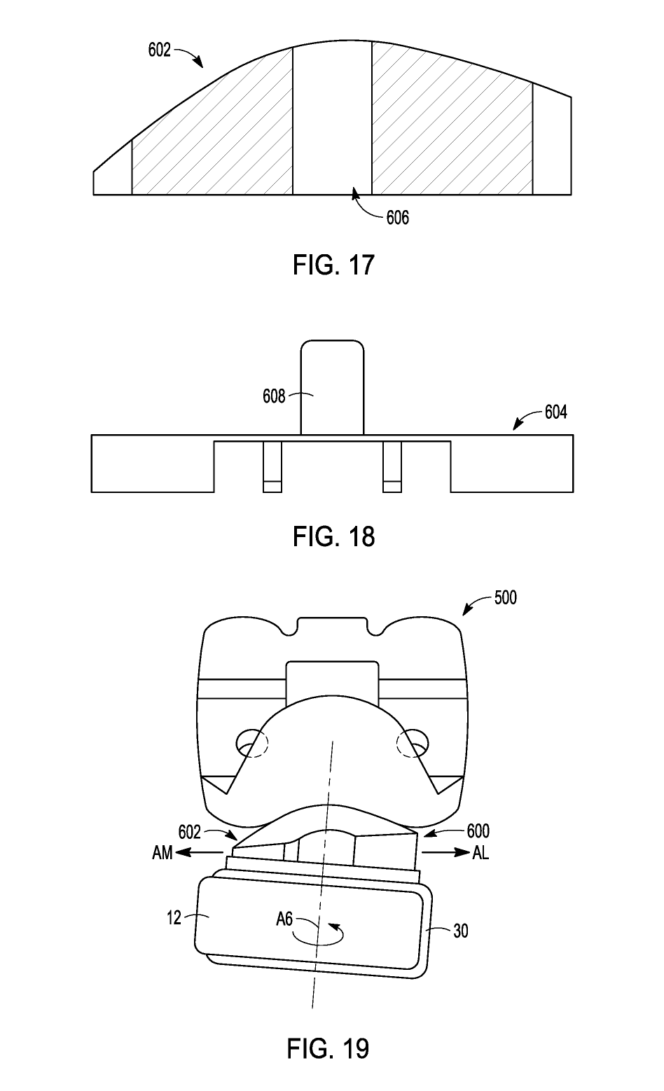

FIG. 16 shows a fifth patellar implant 600 according to another example. The fifth patellar implant 600 can combine aspects of the first patellar implant 26 and the fourth patellar implant 400 and can be configured for translational movement (e.g., medial-lateral movement) and rotational movement relative to the femoral prosthesis and patella. In particular, the fifth patellar implant 600 includes an articular component 602 shown in FIGS. 16 and 17 that can couple with the member 30 (shown in reference to FIGS. 2-4D) via a modular component 604 (FIGS. 16 and 18). The construction of the articular component 602 has been previously described, and therefore, will not be described in great detail. In the example of FIGS. 16 and 17, the articular component 602 can have the non-symmetrical shape as previously described with reference to the fourth tibial patellar 400. As such, the articular component 602 can include a passage 606 therein.

The modular component 604 can be configured to couple with the member 30 using the connection mechanism 58 previously described. As such, the modular component 604 can be capable of translational movement on the member 30 relative to the patella and the femoral prosthesis as previously described. Although not illustrated in the example of FIGS. 16 and 18, the modular component 604 can be tilted or angled (i.e. wedge shaped) in some examples. This can result in certain portions of the modular component 604 having a different thickness relative to other portions. The angle design of the modular component 604 can angle or tilt the articular component 602 relative to the patella and the femoral prosthesis in a similar manner to the non-symmetrical geometry of the articular component in the fourth patellar implant 400 previously described.

The modular component 604 can additionally include a post 608 that can be received in the passage 606 of the articular component 602. The articular component 602 can rotate about the post 608 in the manner of the pegs previously described. Therefore, like the third patellar implant 300 and the fourth patellar implant 400, the fifth patellar implant 600 (in particular the articular component 602) can rotate relative to the patella and femoral prosthesis about the post 608 during kinematic range of motion testing.

FIG. 19 illustrates both rotation (about axis A6) and translation (indicated by arrows AL and AM) of the articulation component 602 of the fifth patellar implant 600 relative to the member 30, the patella 12 and the femoral prosthesis 500. The articular component 602 can also rotate about axis A6 relative to the modular component 604.

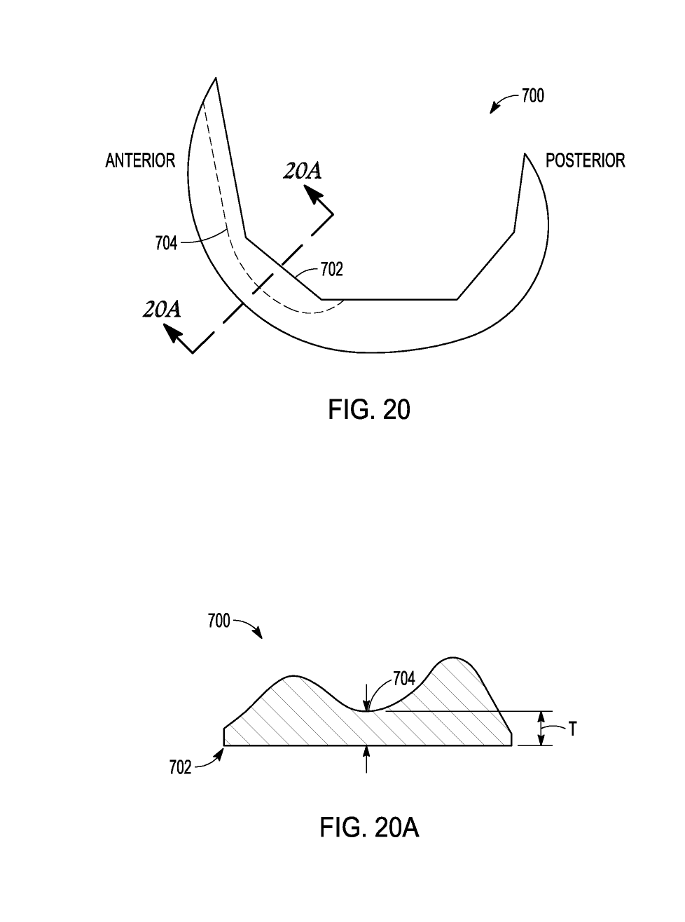

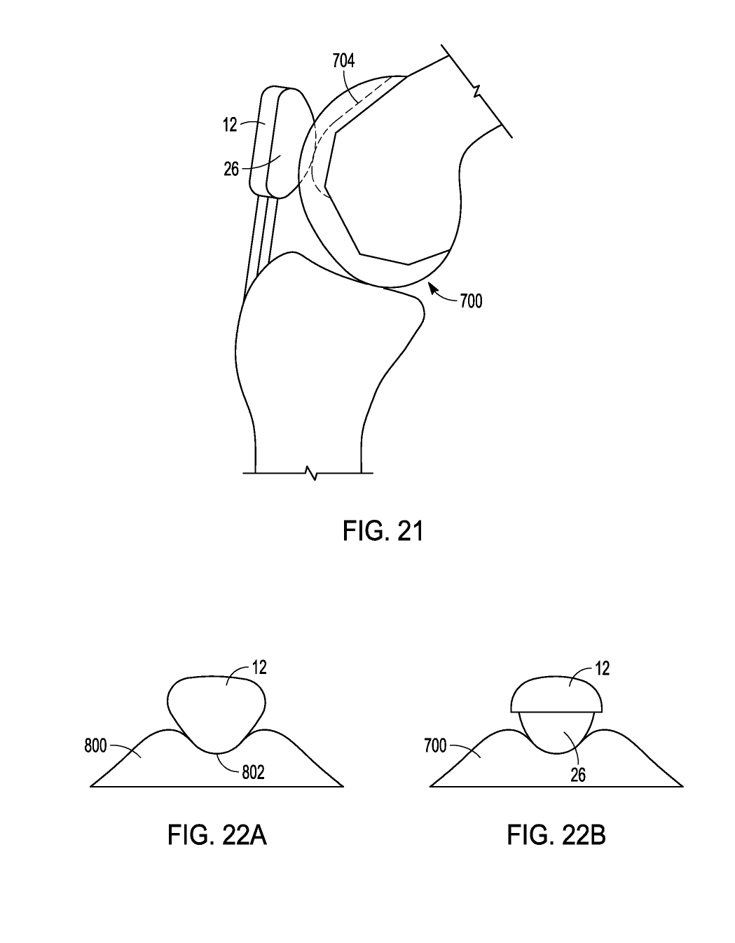

FIGS. 20-22B show an osteotomy method according to one example. FIGS. 20 and 20A illustrate a femoral prosthesis 700 similar to those previously illustrated and described. The femoral prosthesis 700 can have an anterior chamfer 702 and a sulcus 704 (indicated in dashed). FIG. 20A shows a cross section of the femoral prosthesis 700 at the anterior chamfer 702 and further shows a thickness T of the femoral prosthesis 700 as measured at the sulcus 704. FIG. 21 shows a side sagittal view of the patella 12 as previously described and any one of the aforementioned patellar implants (called out as 26 for reference) interfacing with the femoral prosthesis 700. As shown in FIG. 21, the patellar implant 26 is configured to articulate with the femoral component 700 including along the sulcus 704 during movement of the knee joint. FIG. 22A shows the patella 12 and a natural femur 800 in the region of the anterior chamfer prior to osteotomy/resection. Various distances such as the thickness of a saw blade making a resection to the femur 800 and a thickness of the patella 12 as measure to the sulcus 802 are indicated. FIG. 22B shows how these distances are matched once osteotomy of the patella 12 and resection along with implantation of the patellar implant 26 and the femoral prosthesis 700 is performed.

According to one example illustrated in FIGS. 20-22B a method of preparing a knee joint for a patellar implant is disclosed. The method can include measuring a position of a patella within the knee joint including measuring a thickness of the patella, resecting a femur, measuring an amount of femur removed by resecting the femur from an anterior chamfer and further adding a thickness of the saw blade to the amount to obtain a first patellar groove thickness, implanting a desired size of femoral prosthesis on the femur after resecting the femur, measuring a thickness of the femoral prosthesis in a same location of the anterior chamfer to obtain a second patellar groove thickness, determining an amount of bone to be removed from the patella by compensating for the difference between the first patellar groove thickness and the second patellar groove thickness, and after determining the amount of bone to be removed from the patella, resecting the patella to an appropriate thickness to receive the patella implant.

According to some examples, another method of preparing the knee joint for a patellar implant is disclosed. The method can include measuring a position of a patella within the knee joint including measuring a thickness of the patella, resecting a femur, resecting the patella, after resecting the patella, selecting the femoral prosthesis and the patellar implant that provide a desired total thickness for the combination of the femoral prosthesis and the patellar implant. The desired total thickness can be determined by measuring an amount of femur removed by resecting the femur from an anterior chamfer and further adding a thickness of the saw blade to the amount to obtain a first patellar groove thickness, measuring a thickness of the femoral prosthesis in a same location of the anterior chamfer to obtain a second patellar groove thickness, and selecting a thickness of the patellar implant to an amount of bone resected from the patella as well as any difference between the first patellar groove thickness and the second patellar groove thickness. The selecting the thickness of the patellar implant to the amount of bone resected from the patella can be matching according to some examples.

According some examples, the method can include determining a desired thickness for the patellar implant based upon kinematic tests including a range of motion of a trial implant with along a patello-femoral joint between the trail implant and the femoral prosthesis. The trial implant can include an articulation component that is configured to be attachable to and detachable from one or more of a member and the patella.

In some examples, resecting the patella can include performing an osteotomy of the patella to form a substantially planar osteotimized surface that can be angled medial-to-lateral such that at least one of a thickness of the patella at a medial edge is greater than a thickness of the patella at a lateral edge or the thickness of the patella at the medial edge is greater than the thickness of the patella at the lateral edge.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above detailed description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the detailed description as examples or embodiments, with each claim standing on its own as a separate example, and it is contemplated that such examples can be combined with each other in various combinations or permutations. The scope of the invention should be determined with reference to the appended claims, along with the full scope of equivalents to which such claims are entitled.

The claimed invention is:

1. A patellar implant comprising:
a member configured to engage a osteotimized surface of a patella;
an articulation component having an articulation surface configured to articulate with a femoral prosthesis along a patellar groove; and
a connection mechanism connecting the articulation component with the member, the connection mechanism configured to allow for at least one of removal and translational movement of the articulation component relative to the member and patella.

2. The implant of claim 1, wherein the connection mechanism is further configured to allow for rotational movement of the articulation component relative to at least one of the member and patella.

3. The implant of claim 1, wherein the member defines a slot configured to receive the connection mechanism therein, the slot configured to extend generally medial-lateral along the member from a first end to a second end when the member is connected to the patella.

4. The implant of claim 1, wherein the articulation surface is symmetrically shaped as at least one of a spherical dome, a contoured dome or a conical dome.

5. The implant of claim 1, wherein the connection mechanism comprises one or more finger projections extending from an edge of the articulation component, and wherein the member defines one or more grooves configured to receive the one or more finger projections therein.

6. The implant of claim 5, wherein the articulation component comprises a trial prosthetic and is one of a plurality of articulation components, each of the plurality of articulation components configured to attachable and detachable in a substitutable manner, each of the plurality of articulation components configured to create a different thickness for the patellar implant when attached.

7. The implant of claim 1, wherein the connection mechanism comprises a post extending from an opposing side of the articulation component from the articulation surface, and wherein the member defines a slot having a plurality of ridges configured to receive the post therein.

8. A patellar implant comprising:
an articulation component having an articulation surface configured to articulate with a femoral prosthesis at a patello-femoral joint;
a member configured to engage a osteotimized surface of a patella, the member configured to connect to the articulation component;
wherein the articulation component is non-symmetrically shaped about a geometric center of the articulation component and is configured for rotational movement about the member and relative to the patella and the femoral prosthesis to create a variable angle relative to a sulcus of the femoral prosthesis.

9. The patellar implant of claim 8, wherein at least one of the member, a modular component and the articulation component are configured to allow for translational movement of the articulation component relative to the member, the femoral prosthesis and the patella.

10. The patellar implant of claim 8, further comprising a second articulation component configured to connect to and cover the articulation component and interface with the femoral component.

11. A method of preparing a knee joint for a patellar implant, the method comprising:
measuring a position of a patella within the knee joint including measuring a thickness of the patella;
resecting a femur;
measuring an amount of femur removed by resecting the femur from an anterior chamfer and further adding a thickness of the saw blade to the amount to obtain a first patellar groove thickness;
implanting a desired size of femoral prosthesis on the femur after resecting the femur;
measuring a thickness of the femoral prosthesis in a same location of the anterior chamfer to obtain a second patellar groove thickness;
determining an amount of bone to be removed from the patella by compensating for the difference between the first patellar groove thickness and the second patellar groove thickness; and
after determining the amount of bone to be removed from the patella, resecting the patella to an appropriate thickness to receive the patella implant.

12. The method of claim 11, further comprising determining an desired thickness for the patellar implant based upon kinematic tests including a range of motion of a trial implant with along a patello-femoral joint between the trail implant and the femoral prosthesis, wherein the trial implant includes an articulation component that is configured to be attachable to and detachable from a member thereof.

13. The method of claim 11, wherein resecting the patella includes performing an osteotomy of the patella to form a substantially planar osteotimized surface that is angled medial-to-lateral such that at least one of a thickness of the patella at a medial edge is greater than a thickness of the patella at a lateral edge or the thickness of the patella at the medial edge is greater than the thickness of the patella at the lateral edge.

14. The method of claim 11, further comprising:
coupling the patellar implant to the patella, the patellar implant comprising:
a member configured to engage a osteotimized surface of a patella;
an articulation component having an articulation surface configured to articulate with a femoral prosthesis along a patellar groove; and
a connection mechanism connecting the articulation component with the member, the connection mechanism configured to allow for at least one of removal and translational movement of the articulation component relative to the member and patella.

15. The method of claim 14, wherein the patellar implant is configured to allow for rotational movement of the articulation component relative to at least one of the member and patella.

16. The method of claim 11, further comprising:
coupling the patellar implant to the patella, the patellar implant comprising:
an articulation component having an articulation surface configured to articulate with a femoral prosthesis at a patello-femoral joint;
a member configured to engage a osteotimized surface of a patella, the member configured to connect to the articulation component;
wherein the articulation component is non-symmetrically shaped about a geometric center of the articulation component and is configured for rotational movement about the member and relative to the patella and the femoral prosthesis to create a variable angle relative to a sulcus of the femoral prosthesis.

17. The method of claim 16, wherein at least one of the member, a modular component and the articulation component are configured to allow for translational movement of the articulation component relative to the member, the femoral prosthesis and the patella.

18. The method of claim 16, further comprising a second articulation component configured to connect to and cover the articulation component and interface with the femoral component.

19. A method of preparing a knee joint for a patellar implant, the method comprising:
measuring a position of a patella within the knee joint including measuring a thickness of the patella;
resecting a femur;
resecting the patella;
after resecting the patella, selecting the femoral prosthesis and the patellar implant that provide a desired total thickness for the combination of the femoral prosthesis and the patellar implant;
wherein the desired total thickness is determined by:
measuring an amount of femur removed by resecting the femur from an anterior chamfer and further adding a thickness of the saw blade to the amount to obtain a first patellar groove thickness;
measuring a thickness of the femoral prosthesis in a same location of the anterior chamfer to obtain a second patellar groove thickness;
selecting a thickness of the patellar implant to an amount of bone resected from the patella as well as any difference between the first patellar groove thickness and the second patellar groove thickness.
